(19) United States
(12) Patent Application Publication
(10) Pub. No.: US 2020/0286583 A1
(43) Pub. Date: Sep. 10, 2020

(54) COPY NUMBER MEASUREMENT DEVICE, COMPUTER READABLE MEDIUM, COPY NUMBER MEASUREMENT METHOD AND GENE PANEL

(71) Applicants: Mitsubishi Space Software Co., Ltd., Tokyo (JP); NATIONAL UNIVERSITY CORPORATION HOKKAIDO UNIVERSITY, Sapporo-shi, Hokkaido (JP)

(72) Inventors: Shigeki TANISHIMA, Amagasaki-shi (JP); Ryo MORI, Amagasaki-shi (JP); Keisuke SAKAYORI, Amagasaki-shi (JP); Hiroshi NISHIHARA, Sapporo-shi (JP); Sayaka YUZAWA, Sapporo-shi (JP)

(73) Assignees: Mitsubishi Space Software Co., Ltd., Tokyo (JP); NATIONAL UNIVERSITY CORPORATION HOKKAIDO UNIVERSITY, Sapporo-shi, Hokkaido (JP)

(21) Appl. No.: 16/645,746

(22) PCT Filed: Sep. 10, 2018
(86) PCT No.: PCT/JP2018/033424

§ 371 Date: Mar. 9, 2020

(30) Foreign Application Priority Data
Sep. 13, 2017 (JP) 2017-175703

(51) Int. Cl. G16B 20/10; G16B 20/20

(52) U.S. Cl. CPC G16B 20/10; G16B 20/20

(57) ABSTRACT

A position identification unit (110) maps a plurality of tumor sample reads to a human genome sequence, and identifies, for each target gene, a target position which is a genome position of a base, the genome position having changed with respect to the human genome sequence. A frequency calculation unit (120) calculates a variant allele frequency for each target position of each target gene. A distance calculation unit (130) calculates, for each target gene, a feature distance equivalent to a difference between a variant allele frequency corresponding to a peak density and a reference variant allele frequency in a density distribution indicating a density of the number of mapping reads with respect to the variant allele frequency. A coefficient calculation unit (140) calculates a correction coefficient using the feature distance of each target gene. A copy-number calculation unit (150) calculates the copy number of each target gene in the cancer cell using the copy number per target gene in a tumor sample and a correction coefficient.

### TECHNICAL FIELD

The present invention relates to a technique for measuring the accurate copy number in a target sequence.

### BACKGROUND ART

There is a service called clinical sequence that examines a gene mutation in a cancer patient and provides optimal treatment.

Sequence is to read bases of a genetic material and learn a sequence indicating genetic information of the genetic material.

Sequence types include whole genome sequence, whole exome sequence, and target sequence.

Whole genome sequence is a sequence performed on the whole genome including a region where no gene exists.

Whole exome sequence is a sequence performed on gene regions.

Target sequence is a sequence performed on some genes. Specifically, target sequence is performed on genes related to cancer.

Condition of a cancer patient may worsen, and accordingly it is desired that a test result can be obtained in a short time. Since the clinical sequence is not covered by insurance, the entire cost is borne by the patient.

Therefore, in the clinical sequence, a comparative analysis is performed by target sequence being a sequence that can be performed on a daily basis. This leads to time reduction and cost reduction.

In comparative analysis, a normal sample that is not cancer and a tumor sample are used. Specifically, blood is used as a normal sample that is not cancer, and a surgical specimen is used as a tumor sample. Based on the difference between a gene sequence of the normal sample and a gene sequence of the tumor sample, single nucleotide variants (SNVs) derived from cancer and copy number variations (CNVs) are detected. When the gene sequence of the tumor sample is compared with the gene sequence of the normal sample, variants resulting from an individual difference are excluded, so that only a cancer-derived mutation can be learned. The comparative analysis is also called differential analysis.

Prior to CNV detection, multiple reads are obtained from each sample, and the reads are mapped to a human genome sequence.

The number of reads mapped to a target gene region in the human genome sequence approximates the number of chromosomes containing the target gene in an actual cell. Therefore, the copy number of chromosome in the cell can be estimated based on the number of mapped reads.

In CNV detection, if the normalized number of reads from a gene in a cancer cell is larger than the normalized number of reads from a gene in a normal cell, it is determined that the gene is amplified in the cancer cell. If the read number of a gene in a cancer cell is smaller than the number of reads from a gene in a normal cell, it is determined that the gene is decreased in the cancer cell.

Usually, a human gene exists in 2 copies. Therefore, when reads 1.5 times as many as the standard are mapped to a gene region, it is determined that this gene exists in 3 copies.

Non-Patent Literature 1 and Non-Patent Literature 2 are literatures related to micro sequence analysis and disclose a correlation between a Log R Ratio (LRR) and a B Allele Frequency (BAF).

Non-Patent Literature 3 discloses that a phenomenon where the copy number of the short arm of chromosome 1 and the copy number of the long arm of chromosome 19 are both decreased is an important factor that affects the prognosis of a brain tumor.

### CITATION LIST

### Patent Literature

Non-Patent Literature 1: Cathy C. L, et al. Detectable clonal mosaicism from birth to old age and its relationship to cancer, Nature Genetics Volume 44, June 2012, pp. 642-650
Non-Patent Literature 2: C Alkan, et al. Genome Structural variation discovery and genotyping, Nature Reviews Genetics 12, May 2011, pp. 363-376
Non-Patent Literature 3: Louis D N, et al. Acta Neuropathol. June 2016, 131 (6): 803-20. doi: 10.1007/s00401-016-1545-1.

### SUMMARY OF INVENTION

### Technical Problem

CNV detection in the target sequence has the following problems.

Usually, in CNV detection, among ratios of the number of reads (to be referred to as “read number ratios” hereinafter) from genes in a cancer cell to the number of reads from genes in a normal cell of the respective regions, the ratio of the number of read having the highest frequency is treated as the ratio of the number of read at which mapping to a 2-copy region is performed.

Even if the copy number of some genes is increased or decreased, the average copy number is 2 copies in the whole genome because the copy numbers of the other genes are 2 copies. That is, in the case of whole genome sequence performed on the whole genome, the frequency of the read number ratio at which mapping to a 2-copy region is performed is the highest. Therefore, the accurate copy number can be obtained by ordinary CNV detection.

On the other hand, a gene related to cancer is likely to be amplified or decreased. Therefore, in target sequence performed on a gene related to cancer, there is a possibility that the average copy number is not 2 copies. That is, in the case of target sequence, the frequency of the ratio of the number of read at which mapping to the 2-copy region is performed is not always the highest. Hence, there is a possibility that the accurate copy number cannot be obtained by ordinary CNV detection.

An objective of the present invention is to be able to obtain the accurate copy number in target sequence.

### Solution to Problem

A copy-number measurement device according to the present invention includes:

a position identification unit to map a plurality of tumor sample reads which are a plurality of reads obtained from a tumor sample involving a cancer cell, to a human genome sequence, and identify, for each target gene, a target position which is a genome position of a base, the genome position having changed with respect to the human genome sequence;

a frequency calculation unit to calculate a variant allele frequency for each target position of each target gene;

a distance calculation unit to calculate, for each target gene, a feature distance equivalent to a difference between a variant allele frequency corresponding to a peak density and a reference variant allele frequency in a density distribution indicating a density of the number of mapping reads with respect to the variant allele frequency, the number of mapping reads being a number of tumor sample reads mapped to respective target positions in the target gene;

a coefficient calculation unit to calculate a correction coefficient being used for correcting the copy number of each target gene in the tumor sample, using the feature distance of each target gene; and

a copy-number calculation unit to calculate the copy number of each target gene in the cancer cell using the copy number of each target gene in the tumor sample and the correction coefficient.

The distance calculation unit generates a scatter graph indicating a relation between a variant allele frequency of each target position and the mapping read number of each target position; converts the scatter graph to a density distribution graph; generates a correlation graph indicating a correlation between a lower area and a upper area, the lower area being, of the density distribution graph, a region expressing a variant allele frequency that is equal to or lower than the reference variant allele frequency, the upper area being, of the density distribution graph, a region expressing a variant allele frequency that is equal to or higher than the reference variant allele frequency; and calculates, as the feature distance, an absolute value of a difference between a variant allele frequency corresponding to a peak correlation value and the reference variant allele frequency, in the correlation graph.

The correlation graph indicates a correlation in density between a variant allele frequency in the lower area and a variant allele frequency in the upper area that are equal to each other regarding absolute values of differences thereof from the reference variant allele frequency.

The coefficient calculation unit calculates a value corresponding to a deviation amount between a relation graph and a measurement point, as the correction coefficient, the relation graph indicating a relation between the feature distance and a logarithmic value of a ratio of the copy number of a gene in a cancer cell to the copy number of a gene in a normal cell, the measurement point indicating a feature distance of a target gene, and a logarithmic value of a ratio of the copy number of the target gene in the tumor sample to the copy number of the target gene in a normal sample.

The copy-number measurement device includes:

a content ratio calculation unit is provided to calculate a content ratio of the cancer cell in the tumor sample based on the copy number of each target gene in the cancer cell.

The content ratio calculation unit calculates a content ratio candidate using the copy number in the cancer cell for each target gene, and determines the content ratio of the cancer cell in the tumor sample based on the content ratio candidate of each target gene.

The tumor sample is a sample of a brain tumor, and the target gene is at least one of ATRX, IDH1, IDH2, TP53, TERT, BRAF, PDGFRA, MET, EGFR, BRSK1, EHD2, AKT2, TP73, NMNAT1, TGFBR3, and PTEN.

A copy-number measurement program of the present invention causes a computer to function as:

a position identification unit to map a plurality of tumor sample reads which are a plurality of reads obtained from a tumor sample involving a cancer cell, to a human genome sequence, and identify, for each target gene, a target position, which is a genome position of a base, the genome position having changed with respect to the human genome sequence;

a frequency calculation unit to calculate a variant allele frequency for each target position of each target gene;

a distance calculation unit to calculate, for each target gene, a feature distance equivalent to a difference between a variant allele frequency corresponding to a peak density and a reference variant allele frequency in a density distribution indicating a density of the number of mapping reads with respect to the variant allele frequency, the number of mapping reads being a number of tumor sample reads mapped to respective target positions in the target gene;

a coefficient calculation unit to calculate a correction coefficient being used for correcting the copy number of each target gene in the tumor sample, using the feature distance of each target gene; and

a copy-number calculation unit to calculate the copy number of each target gene in the cancer cell using the copy number of each target gene in the tumor sample and the correction coefficient.

The distance calculation unit generates a scatter graph indicating a relation between a variant allele frequency of each target position and the number of mapping reads of each target position; converts the scatter graph to a density distribution graph; generates a correlation graph indicating a correlation between a lower area and a upper area, the lower area being, of the density distribution graph, a region expressing a variant allele frequency that is equal to or lower than the reference variant allele frequency, the upper area being, of the density distribution graph, a region expressing a variant allele frequency that is equal to or higher than the reference variant allele frequency; and calculates, as the feature distance, an absolute value of a difference between a variant allele frequency corresponding to a peak correlation value and the reference variant allele frequency in the correlation graph.

The correlation graph indicates a correlation in density between a variant allele frequency in the lower area and a variant allele frequency in the upper area that are equal to each other regarding absolute values of differences thereof from the reference variant allele frequency.

The coefficient calculation unit calculates a value corresponding to a deviation amount between a relation graph and a measurement point, as the correction coefficient, the relation graph indicating a relation between the feature distance and a logarithmic value of a proportion of the copy number of a gene in a cancer cell to the copy number of a gene in a normal cell, the measurement point indicating a feature distance of a target gene, and a logarithmic value of a proportion of the copy number of the target gene in the tumor sample to the copy number of the target gene in a normal sample.

A content ratio calculation unit is provided to calculate a content ratio of the cancer cell in the tumor sample based on the copy number of each target gene in the cancer cell.

The content ratio calculation unit calculates a content ratio candidate using the copy number in the cancer cell for each target gene, and determines the content ratio of the cancer cell in the tumor sample based on the content ratio candidate of each target gene.

The tumor sample is a sample of a brain tumor, and

the target gene is at least one of ATRX, IDH1, IDH2, TP53, TERT, BRAF, PDGFRA, MET, EGFR, BRSK1, EHD2, AKT2, TP73, NMNAT1, TGFBR3, and PTEN.

A copy-number measurement method includes:

by a position identification unit, mapping a plurality of tumor sample reads which are a plurality of reads obtained from a tumor sample involving a cancer cell to a human genome sequence, and identifying, for each target gene, a target position which is a genome position of a base, the genome position having changed with respect to the human genome sequence;

by a frequency calculation unit, calculating a variant allele frequency for each target position of each target gene;

by a distance calculation unit, calculating, for each target gene, a feature distance equivalent to a difference between a variant allele frequency corresponding to a peak density and a reference variant allele frequency in a density distribution indicating a density of a mapping read number with respect to the variant allele frequency, the mapping read number being a number of tumor sample reads mapped to respective target positions in the target gene;

by a coefficient calculation unit, calculating a correction coefficient being used for correcting the copy number of each target gene in the tumor sample, using the feature distance of each target gene; and

by a copy-number calculation unit, calculating the copy number of each target gene in the cancer cell using the copy number of each target gene in the tumor sample and the correction coefficient.

A gene panel according to the present invention contains a gene set including of all of ATRX, IDH1, IDH2, TP53, TERT, BRAF, PDGFRA, MET, EGFR, BRSK1, EHD2, AKT2, TP73, NMNAT1, TGFBR3, and PTEN.

A gene panel according to the present invention contains a gene set consisting of ATRX, IDH1, IDH2, TP53, TERT, BRAF, PDGFRA, MET, EGFR, BRSK1, EHD2, AKT2, TP73, NMNAT1, TGFBR3, and PTEN.

A gene panel according to the present invention contains a gene set including at least one of ATRX, IDH1, IDH2, TP53, TERT, BRAF, PDGFRA, MET, EGFR, BRSK1, EHD2, AKT2, TP73, NMNAT1, TGFBR3, and PTEN.

### Advantageous Effects of Invention

According to the present invention, the accurate copy number can be obtained in target sequence.

### BRIEF DESCRIPTION OF DRAWINGS

FIG. 1 is a configuration diagram of a copy-number measurement device 100 in Embodiment 1.

FIG. 2 is a flowchart of a copy-number measurement method in Embodiment 1.

FIG. 3 is a flowchart of a position identification process (S110) in Embodiment 1.

FIG. 4 is a diagram illustrating an example of a mutation position in Embodiment 1.

FIG. 5 is a flowchart of a frequency calculation process (S120) in Embodiment 1.

FIG. 6 is a flowchart of a distance calculation process (S130) in Embodiment 1.

FIG. 7 is a flowchart of a model generation process (S132) in Embodiment 1.

FIG. 8 is a diagram illustrating a scatter graph 201 in Embodiment 1.

FIG. 9 is a diagram illustrating a density distribution graph 202 in Embodiment 1.

FIG. 10 is a diagram illustrating a correlation graph 203 in Embodiment 1.

FIG. 11 is a diagram illustrating a feature distance of the correlation graph 203 in Embodiment 1.

FIG. 12 is a diagram illustrating a relation model 210 in Embodiment 1.

FIG. 13 is a diagram illustrating a measurement point group coinciding with the relation model 210 in Embodiment 1.

FIG. 14 is a diagram illustrating a measurement point group not coinciding with the relation model 210 in Embodiment 1.

FIG. 15 is a flowchart of a coefficient calculation process (S140) in Embodiment 1.

FIG. 16 is a flowchart of the coefficient calculation process (S140) in Embodiment 1.

FIG. 17 is a flowchart of a score calculation process (S144) in Embodiment 1.

FIG. 18 is a flowchart of the copy number calculation process (S150) in Embodiment 1.

FIG. 19 is a diagram illustrating examples of copy numbers in a whole genome.

FIG. 20 is a graph illustrating examples of the copy number of chromosome 1, the copy number of chromosome 10, and the copy number of chromosome 19.

FIG. 21 is a configuration diagram of a copy-number measurement device 100 in Embodiment 2.

FIG. 22 is a flowchart of a copy-number measurement method in Embodiment 2.

FIG. 23 is a flowchart of a content ratio calculation process (S160) in Embodiment 2.

### DESCRIPTION OF EMBODIMENTS

In embodiments and drawings, the same elements and equivalent elements are denoted by the same reference numeral. Description of an element denoted by the same reference numeral will be omitted or simplified appropriately. Arrows in the drawings mainly indicate flows of data or flows of process.

### Embodiment 1

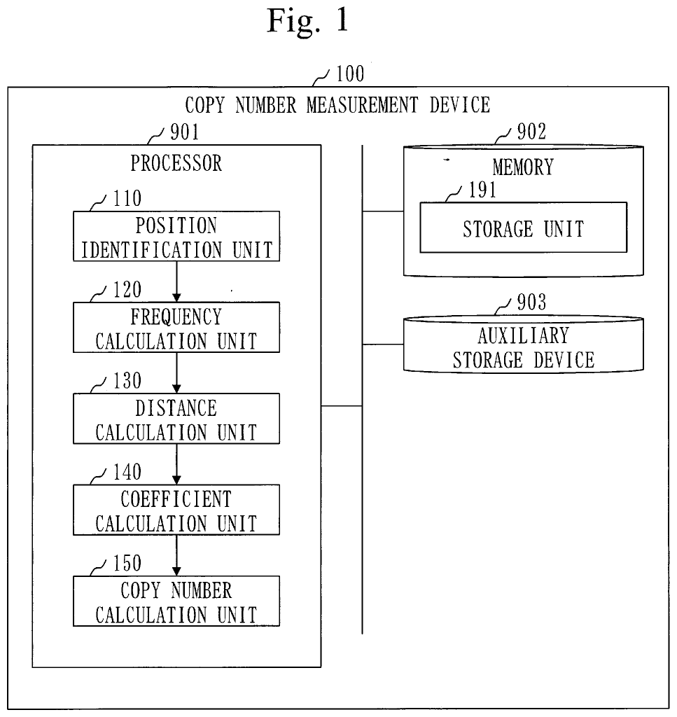

An embodiment for obtaining the accurate copy number in target sequence will be described referring to FIGS. 1 to 18.

***Description of Configuration***

A configuration of a copy-number measurement device 100 will be described referring to FIG. 1.

The copy-number measurement device 100 is a computer provided with hardware devices such as a processor 901, a memory 902, and an auxiliary storage device 903. These hardware devices are connected to each other via a signal line.

The processor 901 is an integrated circuit (IC) which performs arithmetic processing and controls the other hardware devices. The processor 901 is, for example, a central processing unit (CPU), a digital signal processor (DSP), or a graphics processing unit (GPU).

The memory 902 is a volatile storage device. The memory 902 is also called a main storage device or main memory. The memory 902 is, for example, a random access memory (RAM). Data stored in the memory 902 is kept in the auxiliary storage device 903 as necessary.

The auxiliary storage device 903 is a non-volatile storage device. The auxiliary storage device 903 is, for example, a read only memory (ROM), a hard disk drive (HDD), or a flash memory. Data stored in the auxiliary storage device 903 is loaded to the memory 902 as necessary.

The copy-number measurement device 100 is provided with software elements such as a position identification unit 110, a frequency calculation unit 120, a distance calculation unit 130, a coefficient calculation unit 140, a copy-number calculation unit 150, and a content ratio calculation unit 160. The software elements are elements implemented by software.

A copy-number measurement program to cause the computer to function as the position identification unit 110, frequency calculation unit 120, distance calculation unit 130, coefficient calculation unit 140, copy-number calculation unit 150, and content ratio calculation unit 160 is stored in the auxiliary storage device 903. The copy-number measurement program is loaded to the memory 902 and executed by the processor 901.

Furthermore, an operating system (OS) is stored in the auxiliary storage device 903. At least part of the OS is loaded to the memory 902 and executed by the processor 901.

That is, the processor 901 executes the copy-number measurement program while executing the OS.

Data obtained by executing the copy-number measurement program is stored in a storage device such as the memory 902, the auxiliary storage device 903, and a register in the processor 901 or a cache memory in the processor 901.

The memory 902 functions as a storage unit 191 to store data. Alternatively, another storage device may function as the storage unit 191 in place of the memory 902 or along with the memory 902.

The copy-number measurement device 100 may be provided with a plurality of processors that replace the processor 901. The plurality of processors share the role of the processor 901.

The copy-number measurement program can be computer-readably stored in a non-volatile storage medium such as a magnetic disk, an optical disk, and a flash memory. The non-volatile storage medium is a non-transitory tangible medium.

***Description of Operation***

An operation of the copy-number measurement device 100 corresponds to a copy-number measurement method. A procedure of the copy-number measurement method corresponds to a procedure of the copy-number measurement program.

The copy-number measurement method is a method of measuring the copy number of a target gene in a cancer cell.

The target gene is a gene dedicated to prediction of prognosis of brain tumor. The gene dedicated to prediction of prognosis of the brain tumor is a gene whose relation with brain tumor is known, among genes existing in a region where it is possible to determine whether the copy number of a short arm of chromosome 1 and the copy number of a long arm of chromosome 19 are both decreasing.

Specifically, examples of the target gene are ATRX, IDH1, IDH2, TP53, TERT, BRAF, PDGFRA, MET, EGFR, BRSK1, EHD2, AKT2, TP73, NMNAT1, TGFBR3, and PTEN. Alternatively, the target gene is one or more of these genes.

A gene panel in Embodiment 1 contains a gene set including at least one of the target genes mentioned above.

Specifically, the gene set includes all of the target genes mentioned above. Particularly, the gene set consists of the target genes mentioned above.

The gene panel is a tool for analyzing gene mutation. The gene panel is also called a sequence panel.

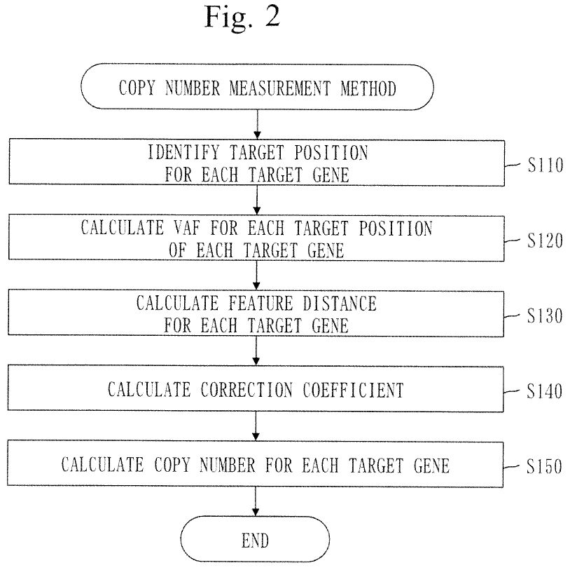

The procedure of the copy-number measurement method will be described referring to FIG. 2.

In step S110, the position identification unit 110 identifies a target position for each target gene.

The target position is a genome position of a base changing with respect to a human genome sequence. Particularly, a genome position that has significantly changed is the target position.

The genome position is a position of a base in the human genome sequence.

Specifically, the position identification unit 110 maps a plurality of tumor sample reads to a human genome sequence. Then, the position identification unit 110 identifies, for each target gene, the target position by comparing the tumor sample reads mapped to a region of the target gene in the human genome sequence with the region of the target gene in the human genome sequence.

The plurality of tumor sample reads are a plurality of reads obtained from a tumor sample.

The tumor sample is part of a tumor. A specific example of the tumor is brain tumor. The tumor sample involves a cancer cell and a normal cell.

A read is a fragmented gene sequence and expressed by a letter sequence (base sequence) indicating an order of bases.

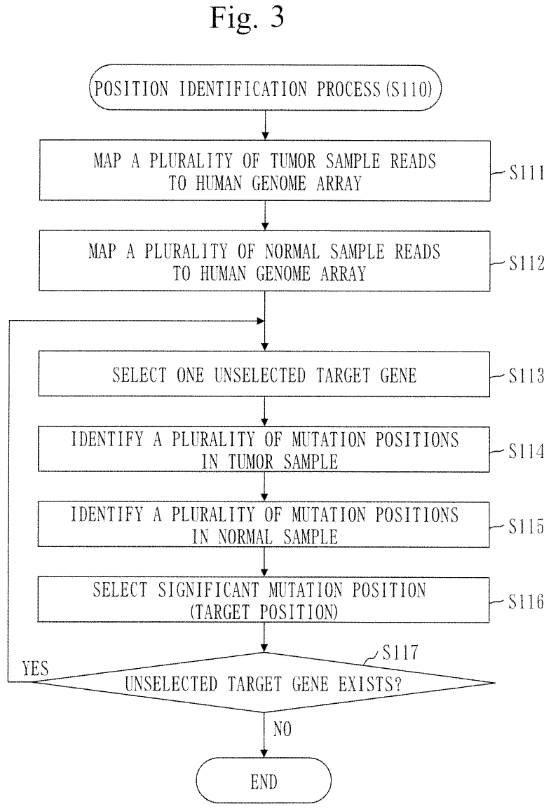

A procedure of a position identification process (S110) will be described referring to FIG. 3.

In step S111, the position identification unit 110 maps the plurality of tumor sample reads to the human genome sequence.

The plurality of tumor sample reads are obtained from the tumor sample by a DNA sequencer and stored in the storage unit 191.

The number of reads obtained by the DNA sequencer is about 100,000. Each read has a length corresponding to 100 bases approximately.

In step S112, the position identification unit 110 maps a plurality of normal sample reads to the human genome sequence.

A normal sample is a portion other than tumor.

The plurality of normal sample reads are obtained from the normal sample by the DNA sequencer and stored in the storage unit 191.

In step S113, the position identification unit 110 selects one unselected target gene.

Processes from step S114 to step S116 are performed on the target gene selected in step S113. In the human genome sequence, a region where the target gene exists is called a target region.

In step S114, the position identification unit 110 compares the bases of the tumor sample reads mapped to the target region with bases of the target region in the human genome sequence.

The position identification unit 110 then identifies a plurality of mutation positions in the tumor sample based on the comparison result.

A mutation position is the genome position of a base changing with respect to the human genome sequence. That is, the mutation position is a genome position of a base of single nucleotide variant (SNV).

A method of identifying the mutation position is the same as the conventional method of identifying a position of a base of SNV.

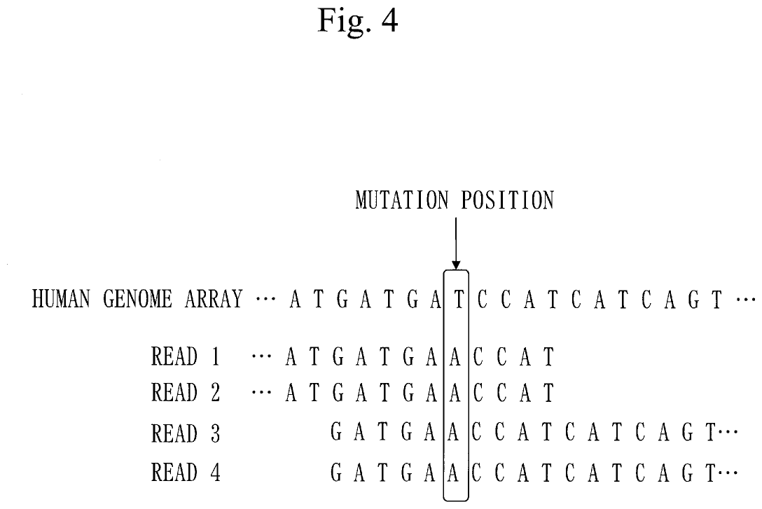

FIG. 4 illustrates how four reads are mapped to a human genome sequence.

Bases (A) in the mapped reads differ from a base “T” in the human genome sequence. That is, the bases of the mapped reads have changed to “A” with respect to the base “T” in the human genome sequence.

Hence, the genome position of the base “T” in the human genome sequence is a mutation position.

Back to FIG. 3, description continues from step S115.

In step S115, the position identification unit 110 compares the bases of the normal sample reads mapped to the target region with the bases of the target region in the human genome sequence.

The position identification unit 110 then identifies a plurality of mutation positions in the normal sample based on the comparison result.

A method of identifying the mutation position is the same as the conventional method of identifying a position of a base of SNV.

In step S116, the position identification unit 110 compares the plurality of mutation positions in the tumor sample with the plurality of mutation positions in the normal sample.

The position identification unit 110 then selects a significant mutation position from among the plurality of mutation positions in the tumor sample based on the comparison result. The significant mutation position is a position of a base significantly changing and is treated as the target position.

Specifically, the position identification unit 110 conducts Fisher's test or another test.

In step S117, the position identification unit 110 determines whether an unselected target gene exists.

If an unselected target gene exists, the process proceeds to step S111.

If an unselected target gene does not exist, the position identification process (S110) ends.

Back to FIG. 2, step S120 will be described.

In step S120, the frequency calculation unit 120 calculates a variant allele frequency (VAF) for each target position of each target gene.

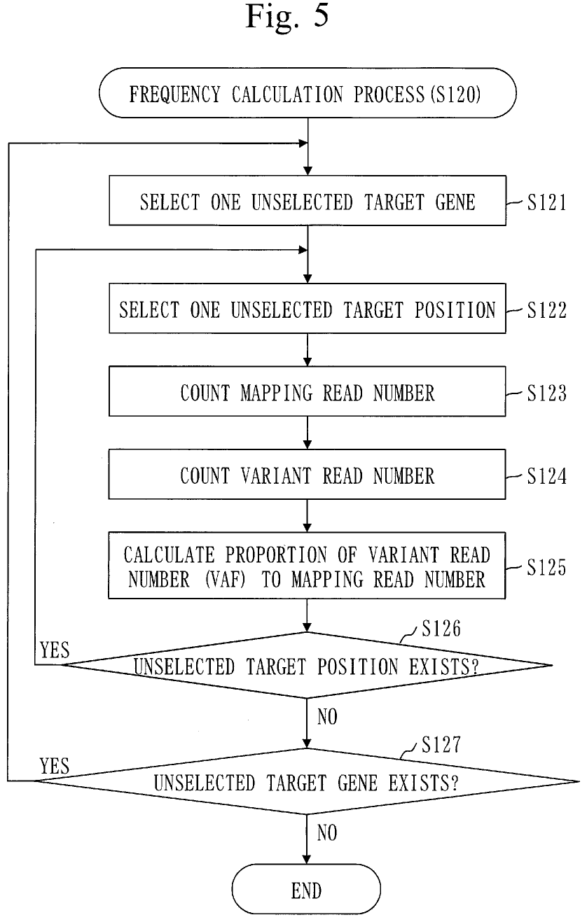

A procedure of frequency calculation process (S120) will be described referring to FIG. 5.

In step S121, the frequency calculation unit 120 selects one unselected target gene.

Processes from step S122 to step S126 are performed on the target gene selected in step S121.

In step S122, the frequency calculation unit 120 selects one unselected target position.

In step S123 to step S125, a target gene signifies the target gene selected in step S121. A target position signifies the target position selected in step S122.

In step S123, the frequency calculation unit 120 counts the number of mapping reads.

The number of mapping reads is the number of reads that are mapped to the region including the target position, among the plurality of tumor sample reads.

The number of mapping reads is called sequence depth.

In step S124, the frequency calculation unit 120 counts the number of variant reads.

The number of variant reads is the number of reads whose bases at target positions differ from bases in the human genome sequence, among the reads mapped to the target positions.

In step S125, the frequency calculation unit 120 calculates a proportion of the number of variant reads to the number of mapping reads. The calculated proportion is the VAF.

In step S126, the frequency calculation unit 120 determines whether an unselected target position exists.

If an unselected target position exists, the process proceeds to step S122.

If an unselected target position does not exist, the process proceeds to step S127.

In step S127, the frequency calculation unit 120 determines whether an unselected target gene exists.

If an unselected target gene exists, the process proceeds to step S121.

If an unselected target gene does not exist, the frequency calculation process (S120) ends.

Back to FIG. 2, step S130 will be described.

In step S130, the distance calculation unit 130 calculates a feature distance for each target gene.

The feature distance is a value equivalent to a difference between a VAF (variant allele frequency) corresponding to a peak density and a reference VAF (=0.5) in a density distribution indicating a density of the mapping read number with respect to the VAF. The feature distance is equivalent to |BAF deviation from 0.5| described in Non-Patent Literature 1.

The mapping read number signifies the number of tumor sample reads mapped to the respective target positions in the target gene.

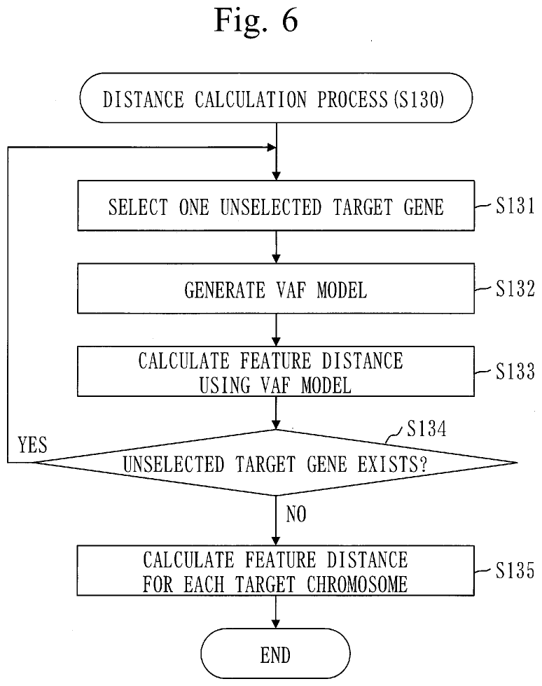

A procedure of a distance calculation process (S130) will be described referring to FIG. 6.

In step S131, the distance calculation unit 130 selects one unselected target gene.

In step S132 and step S133, a target gene signifies the target gene selected in step S131.

In step S132, the distance calculation unit 130 generates a VAF model.

The VAF model is a graph for identifying the VAF corresponding to the peak density.

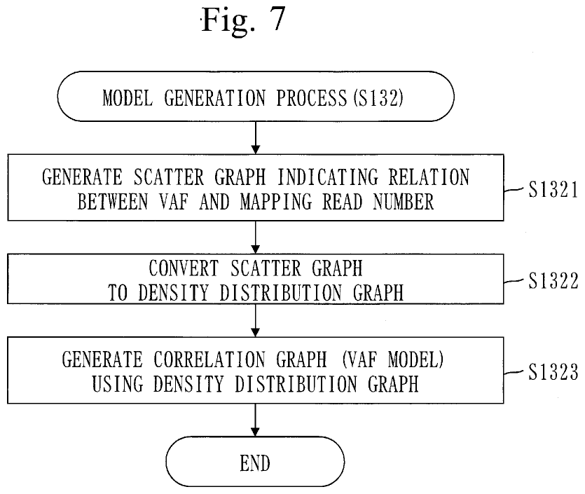

A procedure of a model generation process (S132) will be described referring to FIG. 7.

In step S1321, the distance calculation unit 130 generates a scatter graph indicating a relation between a VAF of each target position and a mapping read number of each target position.

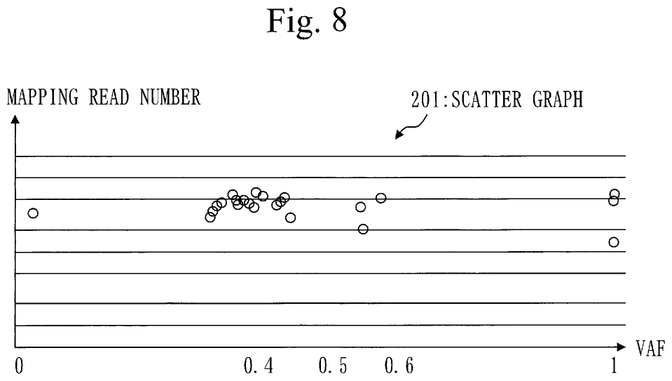

FIG. 8 illustrates a scatter graph 201. The scatter graph 201 is an example of a scatter graph.

In the scatter graph 201, the axis of abscissa represents the VAF, and the axis of ordinate represents the mapping read number.

The scatter graph 201 indicates that a large number of tumor sample reads are mapped to target positions corresponding to VAFs near 0.4. Also, the scatter graph 201 indicates that a certain number of tumor sample reads are mapped to target positions corresponding to VAFs near 0.6 as well.

In step S1322, the distance calculation unit 130 converts the scatter graph to a density distribution graph. The density distribution graph indicates a relation between the VAF and the mapping density.

The mapping density is the density of the mapping read number with respect to the VAF.

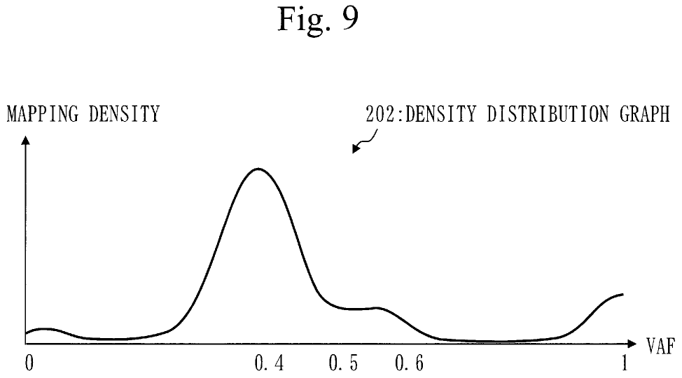

FIG. 9 illustrates a density distribution graph 202. The density distribution graph 202 is a density distribution graph obtained by converting the scatter graph 201 of FIG. 8.

In the density distribution graph 202, the axis of abscissa represents the VAF, and the axis of ordinate represents the mapping density.

The density distribution graph 202 indicates that a mapping density corresponding to a VAF near 0.4 is high. Furthermore, the density distribution graph 202 indicates that a mapping density corresponding to a VAF near 0.6 is also high to a certain degree.

In step S1323, the distance calculation unit 130 generates a correlation graph using the density distribution graph. The generated correlation graph is the VAF model.

The correlation graph indicates a correlation between a lower area of the density distribution graph and a upper area of the density distribution graph. The lower area is a region expressing a VAF that is equal to or lower than the reference VAF (=0.5). The upper area is a region expressing a VAF that is equal to or higher than the reference VAF.

Specifically, the correlation graph indicates a correlation in density between a VAF in the lower area and a VAF in the upper area that are equal to each other regarding absolute values of their differences from the reference VAF.

The distance calculation unit 130 generates the correlation graph as follows. First, taking the reference VAF (=0.5) in the density distribution graph as an axis of target, the distance calculation unit 130 maps a graph of the upper area (VAF>0.5) to the graph of the lower area (VAF<0.5) line-symmetrically.

The distance calculation unit 130 finds a correlation value indicating a correlation between the original graph and the mapped graph in the lower area.

The distance calculation unit 130 generates a correlation graph indicating a relation between VAF and the correlation value in the lower area.

Then, taking the reference VAF as the axis of target, the distance calculation unit 130 maps the lower area to the upper area line-symmetrically.

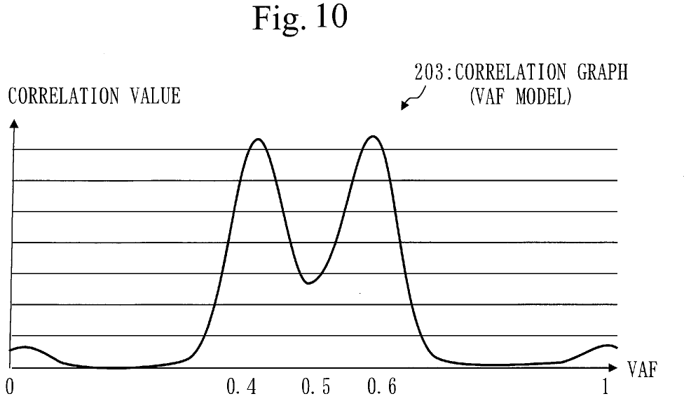

FIG. 10 illustrates a correlation graph 203. The correlation graph 203 is a correlation graph (VAF model) generated with using the density distribution graph 202 of FIG. 9.

In the correlation graph 203, the axis of abscissa represents the VAF, and the axis of ordinate represents the correlation value.

The correlation graph 203 illustrates that a correlation value corresponding to a VAF near 0.4 and a correlation value corresponding to a VAF near 0.6 are both peaks of the correlation values.

Back to FIG. 6, description continues from step S133.

In step S133, the distance calculation unit 130 calculates the feature distance using the VAF model.

Specifically, the distance calculation unit 130 calculates an absolute value of a difference between a VAF (variant allele frequency) corresponding to the peak correlation value and the reference VAF (=0.5) in the VAF model (correlation graph). The calculated absolute value is the feature distance.

A peak correlation value is the peak of the correlation value in the VAF model.

When a plurality of peak correlation values exist, the distance calculation unit 130 finds the feature distance using a VAF corresponding to a maximum peak correlation value.

For example, the distance calculation unit 130 identifies the VAF corresponding to the peak correlation value as follows.

The distance calculation unit 130 performs the following process for each set of a target VAF, a low VAF, and a high VAF while changing the target VAF. The low VAF is a VAF smaller than the target VAF by a predetermined value. The high VAF is a VAF larger than the target VAF by a predetermined value.

First, the distance calculation unit 130 finds a first straight line connecting a correlation value of the low VAF and a correlation value of the target VAF.

Furthermore, the distance calculation unit 130 finds a second straight line connecting the correlation value of the target VAF and a correlation value of the high VAF.

The distance calculation unit 130 finds a gradient of the first straight line and a gradient of the second straight line.

The distance calculation unit 130 compares a sign of the gradient of the first straight line with a sign of the gradient of the second straight line.

If the sign of the gradient of the first straight line is different from the sign of the gradient of the second straight line, the distance calculation unit 130 selects the target VAF. The selected target VAF is the VAF corresponding to the peak correlation value.

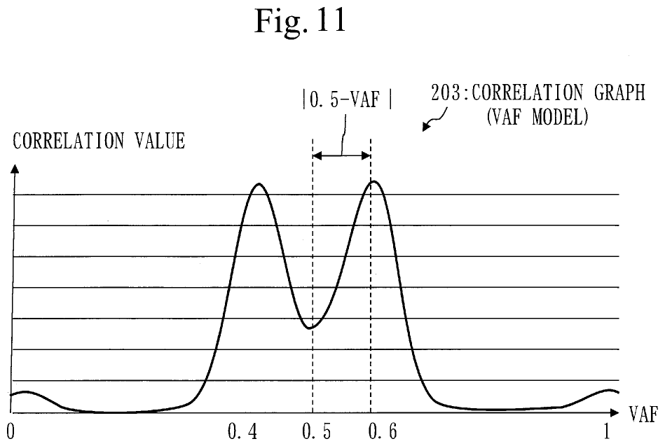

FIG. 11 illustrates a feature distance of the correlation graph 203. Note that |0.5−VAF| expresses the feature distance.

In the correlation graph 203, VAFs corresponding to the peak correlation values are a VAF of approximately 0.4 and a VAF of approximately 0.6. Hence, the feature distance is approximately 0.1.

In step S134, the distance calculation unit 130 determines whether an unselected target gene exists.

If an unselected target gene exists, the process proceeds to step S131.

If an unselected target gene does not exist, the process proceeds to step S135.

In step S135, the distance calculation unit 130 calculates a feature distance for each target chromosome.

The target chromosomes are chromosome 1, chromosome 10, and chromosome 19.

A method of calculating the feature distance of a target chromosome is similar to the method of calculating the feature distance of a target gene.

Back to FIG. 2, step S140 will be described.

In step S140, the coefficient calculation unit 140 calculates a correction coefficient using the feature distance of each target gene.

The correction coefficient is a coefficient for correcting the copy number of the target gene (and target chromosome) in the tumor sample.

By correcting the copy number of the target gene (and target chromosome) in the tumor sample using the correction coefficient, the copy number of the target gene (and target chromosome) in the cancer cell can be obtained.

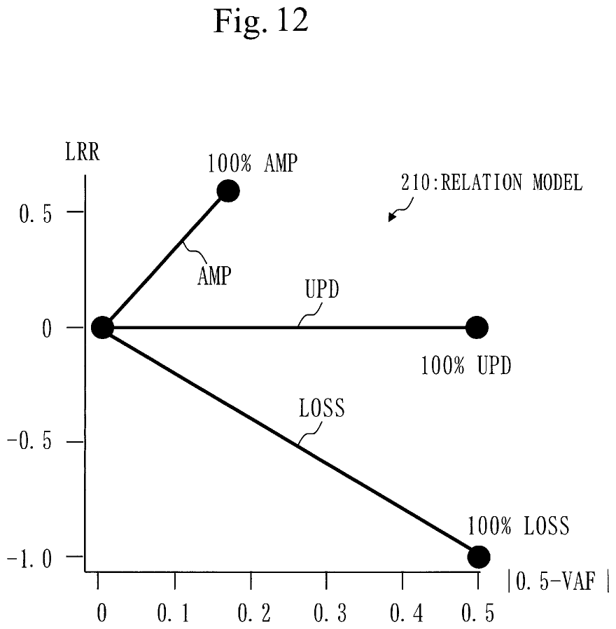

FIG. 12 illustrates a relation model 210.

The relation model 210 indicates a relation between the feature distance and a Log R Ratio (LRR) of the copy number. Note that |0.5−VAF| expresses the feature distance.

The LRR is a value that expresses, by a logarithmic value, a ratio of the copy number of a gene in a cancer cell to the copy number of a gene in a normal cell.

The LRR can be expressed by the following formula.

LRR=log₂(tumor/normal)

Note that tumor represents the copy number of a gene in the cancer cell and normal presents the copy number of a gene in the normal cell. The value of normal is 2.

When tumor is 2, the LRP is 0, so there is a possibility that the state of the gene is uniparental disomy (UPD). UPD is a state where only a mother-derived gene or a father-derived gene exists in 2 copies and thus heterozygosity is lost.

When tumor is less than 2, the LRR is a negative value, and the state of the gene is LOSS. LOSS is a state where a gene is decreased.

When tumor is larger than 2, the LRR is a positive value, and the state of the gene is AMP. AMP is a state where a gene is amplified.

It is known that the feature distance and the LRR of the copy number agree with the relation model 210, as described in Non-Patent Literature 1.

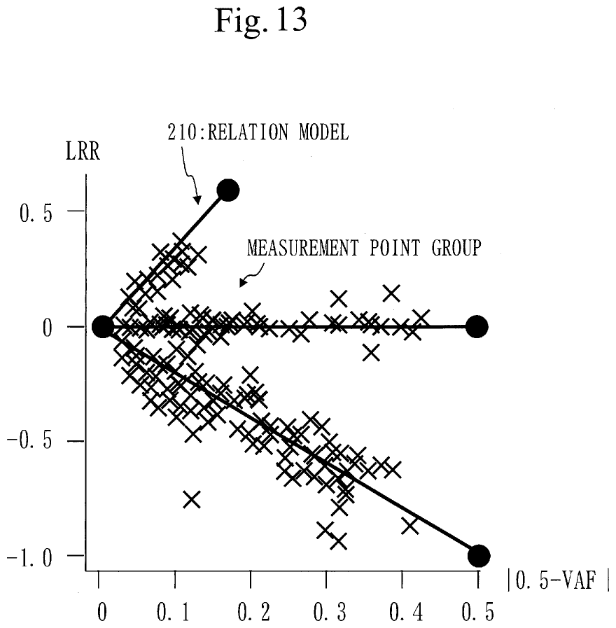

When a feature distance of a gene in the cancer cell and the LRR of a gene in the cancer cell are measured, a graph as illustrated in FIG. 13 is obtained. Each cross mark represents a measurement point.

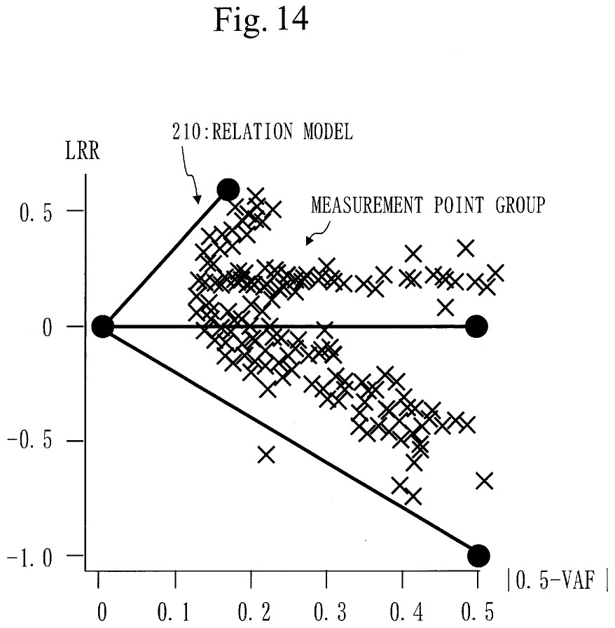

For example, assume that a feature distance of a target gene in a tumor sample and an LRR of the target gene in the tumor sample are measured, and that a graph as illustrated in FIG. 14 is consequently obtained. The LRR of the target gene in the tumor cell is a logarithmic value of a proportion of the copy number of the target gene in the tumor sample to the copy number of the target gene in the normal sample.

The correction coefficient corresponds to a deviation amount of a measurement point group from the relation model 210. That is, when the measurement point group is corrected using the correction coefficient, the corrected measurement point group agrees with the relation model 210, as illustrated in FIG. 13.

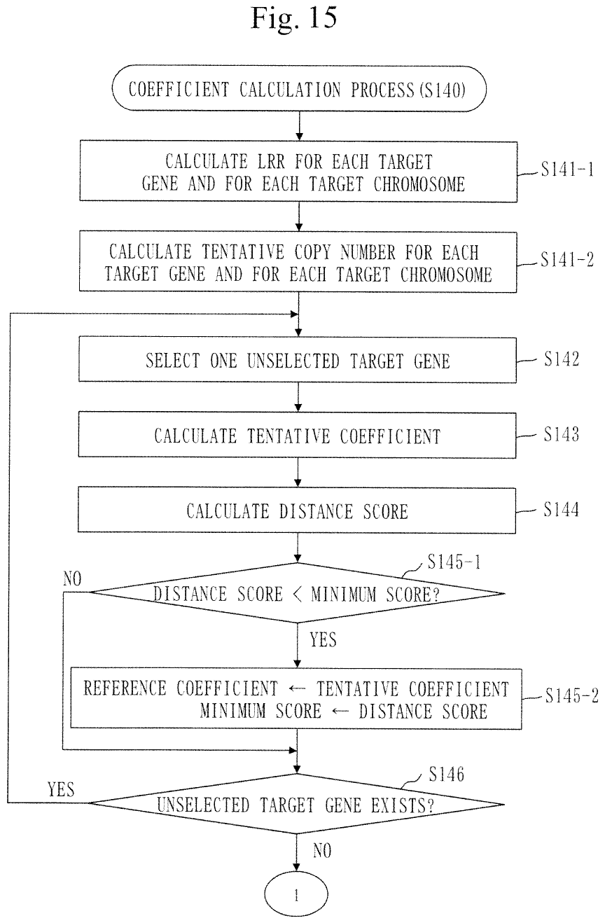

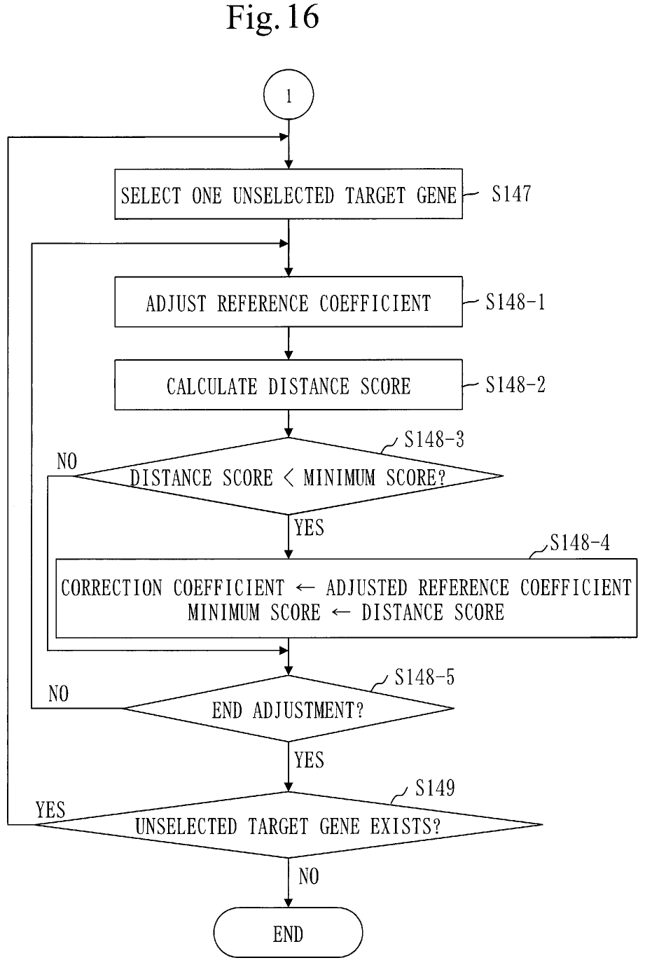

A procedure of a coefficient calculation process (S140) will be described referring to FIGS. 15 and 16.

In step S141-1 (see FIG. 15), the coefficient calculation unit 140 calculates an LRR for each target gene. Furthermore, the coefficient calculation unit 140 calculates an LRR for each target chromosome.

The calculated LRR is a logarithmic value of a proportion of the copy number of the target gene (or target chromosome) in the tumor sample to the copy number of the target gene (or target chromosome) in the normal sample.

The LRR of the target gene (or target chromosome) is calculated based on the proportion of the number of tumor sample reads mapped to the region of the target genes (or target chromosomes) in human genome sequence to the number of normal sample reads mapped to the region of the target genes (or target chromosomes) in human genome sequence. A method employed to calculate the LRR is a conventional technique.

In step S141-2, the coefficient calculation unit 140 calculates a tentative copy number for each target gene. The coefficient calculation unit 140 also calculates a tentative copy number for each target chromosome.

The tentative copy number corresponds to the copy number of the target gene (or target chromosome) in the tumor sample.

Specifically, the coefficient calculation unit 140 selects a tentative copy number formula depending on the LRR of the target gene (or target chromosome) and evaluates the selected tentative copy number formula using the feature distance of the target gone (or target chromosome). Thus, the tentative copy number of the target gene (or target chromosome) is calculated. The tentative copy number formula is a formula for finding a tentative copy number.

In the tentative copy number formulas listed below, CNₜ expresses the tentative copy number of the target gene (or target chromosome), and |0.5−VAF| expresses the feature distance of the target gene (or target chromosome).

When the LRR is a positive value, the tentative copy number formula is as follows.

CNₜ=1/(0.5−|0.5−VAF|)

When the LRR is zero, the tentative copy number formula is as follows.

CNₜ=2.0

When the LRR is a negative value, the tentative copy number formula is as follows.

CNₜ=1/(0.5+|0.5−VAF|)

In step S142, the coefficient calculation unit 140 selects one unselected target gene.

Processes from step S143 to step S145-2 are performed on the target gene selected in step S142.

In step S143, the coefficient calculation unit 140 calculates a tentative coefficient using the tentative copy number of the target gene.

Specifically, the coefficient calculation unit 140 calculates the tentative coefficient Cₜ of the target gene by evaluating the following formula. Note that CNₜ expresses the tentative copy number of the target gene.

Cₜ=2.0/CNₜ

In step S144, the coefficient calculation unit 140 calculates a distance score.

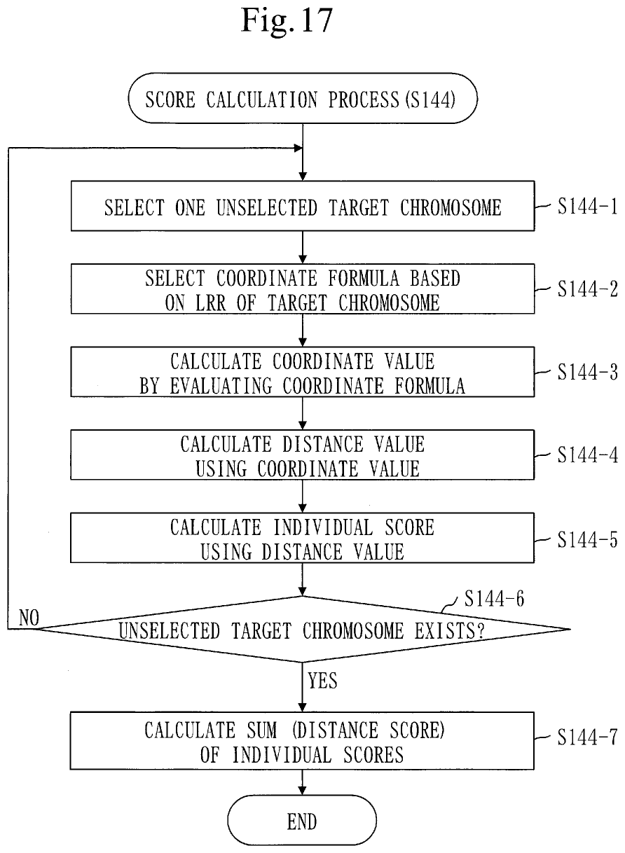

A procedure of a score calculation process (S144) will be explained referring to FIG. 17.

In step S144-1, the coefficient calculation unit 140 selects one unselected target chromosome out of three target chromosomes which are chromosome 1, chromosome 10, and chromosome 19.

Processes from step S144-2 to step S144-5 are performed on the target chromosome selected in step S144-1.

In step S144-2, the coefficient calculation unit 140 selects a coordinate formula depending on the LRR of the target chromosome. The coordinate formula is a formula for finding a coordinate value.

There are three types of coordinate formulas which are a formula for AMP, a formula for UPD, and a formula for LOSS.

AMP signifies amplification of a gene.

UPD signifies uniparental disomy of a gene.

LOSS signifies loss of a gene.

Specifically, the coefficient calculation unit 140 selects a coordinate formula as follows.

When the LRR of the target chromosome is a positive value, the coefficient calculation unit 140 selects a formula for AMP.

When the LRR of the target chromosome is zero, the coefficient calculation unit 140 selects a formula for UPD.

When the LRR of the target chromosome is a negative value, the coefficient calculation unit 140 selects a formula for LOSS.

In step S144-3, the coefficient calculation unit 140 calculates a coordinate value by evaluating the selected coordinate formula.

Specifically, the coefficient calculation unit 140 evaluates the coordinate formula using the tentative coefficient and the tentative copy number of the target chromosome.

In the coordinate formulas below, CNₜ expresses the tentative copy number of the target chromosome, Cₜ expresses the tentative coefficient, and |0.5−VAF| expresses the feature distance of the target chromosome. Also, (x, y) is the coordinate value.

The formula for AMP is:

x=0.5−1/(CNₜ×Cₜ)

y=1/(0.5−|0.5−VAF|)

The formula for UPD is:

x=|0.5−VAF|

y=CNₜ×Cₜ

The formula for LOSS is:

x=1/(CNₜ×Cₜ)−0.5

y=1/(0.5+|0.5−VAF|)

In step S144-4, the coefficient calculation unit 140 calculates an X-direction distance value and a Y-direction distance value using the calculated coordinate value.

Specifically, the coefficient calculation unit 140 calculates an X-direction distance value X % and a Y-direction distance value Y % by evaluating the following formula:

X %=∥0.5−VAF|−x|/x

Y %=|CNt×Ct−y|/|2−y|

In step S144-5, the coefficient calculation unit 140 calculates an individual score using the X-direction distance value and the Y-direction distance value.

Specifically, the coefficient calculation unit 140 calculates an individual score Scoreₙ by evaluating the following formula. Note that m\hat{ }2 signifies a square of m.

Scoreₙ=X %\hat{ }2+Y %\hat{ }2

In step S144-6, the coefficient calculation unit 140 determines whether an unselected target chromosome exists.

If an unselected target chromosome exists, the process proceeds to step S144-1.

If an unselected target chromosome does not exist, the process proceeds to step S144-7.

In step S144-7, the coefficient calculation unit 140 calculates the sum of the individual scores. The sum of the individual scores is the distance score.

Specifically, the coefficient calculation unit 140 calculates the distance score Score by evaluating the following formula. Note that Scoreₙ expresses an individual score of chromosome n.

Score=Score₁+Score₁₀+Score₁₉

Back to FIG. 15, description continues from step S145-1.

In step S145-1, the coefficient calculation unit 140 compares the distance score with the minimum score. The initial value of the minimum score is the maximum value of a variable for a minimum score.

If the distance score is smaller than the minimum score, the process proceeds to step S145-2.

If the distance score is equal to or larger than the minimum score, the process proceeds to step S146.

In step S145-2, the coefficient calculation unit 140 updates the value of a reference coefficient to the value of the tentative coefficient. The initial value of the reference coefficient is 1.

Furthermore, the coefficient calculation unit 140 updates the value of the minimum score to the value of the distance score.

In step S146, the coefficient calculation unit 140 determines whether an unselected target gene exists.

If an unselected target gene exists, the process proceeds to step S142.

If an unselected target gene does not exist, the process proceeds to step S147 (see FIG. 16).

In step S147 (see FIG. 16), the coefficient calculation unit 140 selects one unselected target gene.

Processes from step S148-1 to step S148-5 are performed on the target gene selected in step S147.

In step S148-1, the coefficient calculation unit 140 adjusts the reference coefficient.

Specifically, the coefficient calculation unit 140 selects one unselected adjustment coefficient from an adjustment range and multiplies the reference coefficient by the selected adjustment coefficient.

The adjustment range is a predetermined range and involves a plurality of adjustment coefficients. For example, the adjustment range is a range from 0.80 to 1.20 and involves 41 adjustment coefficients at intervals of 0.01.

A coefficient obtained by adjusting the reference coefficient will be referred to as an adjusted reference coefficient.

In step S148-2, the coefficient calculation unit 140 calculates the distance score using the adjusted reference coefficient. A method of calculating the distance score is similar to the method in step S144 (see FIG. 17) except that the adjusted reference coefficient is used in place of the tentative coefficient.

In step S148-3, the coefficient calculation unit 140 compares the distance score with the minimum score.

If the distance score is smaller than the minimum score, the process proceeds to step S148-4.

If the distance score is equal to or larger than the minimum score, the process proceeds to step S148-5.

In step S148-4, the coefficient calculation unit 140 updates the value of the correction coefficient to the value of the adjusted reference coefficient. The initial value of the correction coefficient is 1.

Furthermore, the coefficient calculation unit 140 updates the value of the minimum score to the value of the distance score.

In step S148-5, the coefficient calculation unit 140 determines whether to end adjustment of the reference coefficient.

Specifically, the coefficient calculation unit 140 determines whether an unselected adjustment coefficient exists within the adjustment range. If an unselected adjustment coefficient does not exist, the coefficient calculation unit 140 ends adjustment of the reference coefficient.

If adjustment of the reference coefficient is to end, the process proceeds to step S149.

If adjustment of the reference coefficient is not to end, the process proceeds to step S148-1.

In step S149, the coefficient calculation unit 140 determines whether an unselected target gene exists.

If an unselected target gene exists, the process proceeds to step S147.

If an unselected target gene does not exist, the coefficient calculation process (S140) ends.

Back to FIG. 2, step S150 will be described.

In step S150, the copy-number calculation unit 150 calculates the copy number of each target gene in the cancer cell using the copy number of each target gene in a tumor sample and the correction coefficient.

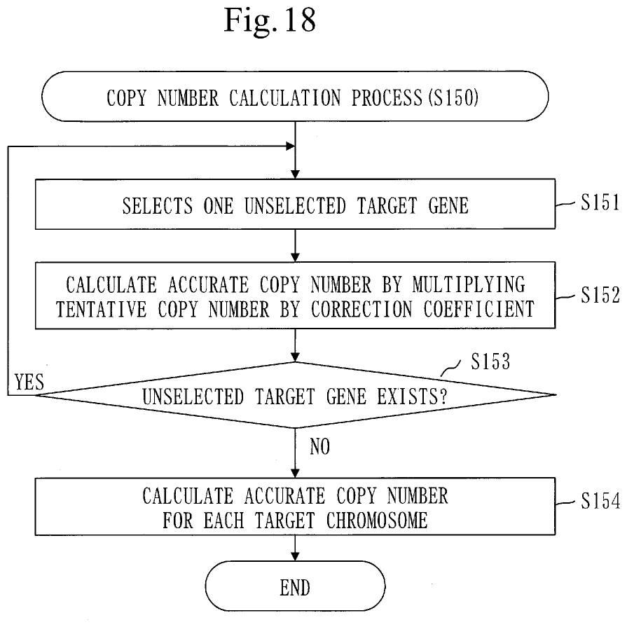

A procedure of the copy-number calculation process (S150) will be described referring to FIG. 18.

In step S151, the copy-number calculation unit 150 selects one unselected target gene.

In step S152, the copy-number calculation unit 150 multiplies the tentative copy number of the target gene by the correction coefficient. The tentative copy number of the target gene is calculated in step S141-2 (see FIG. 15).

The copy number obtained by multiplying the tentative copy number of the target gene by the correction coefficient is the copy number of the target gene in the cancer cell, that is, the accurate copy number of the target gene.

Specifically, the copy-number calculation unit 150 calculates the copy number (CN) by evaluating the following formula. Note that C_{best} expresses a correction coefficient and that CNt expresses a tentative copy number.

CN=C_{best}×CNₜ

In step S153, the copy-number calculation unit 150 determines whether an unselected target gene exists.

If an unselected target gene exists, the process proceeds to step S151.

If an unselected target gene does not exist, the process proceeds to step S154.

In step S154, the copy-number calculation unit 150 calculates the accurate copy number for each target chromosome.

A method of calculating the accurate copy number of the target chromosome is similar to the method of calculating the accurate copy number of the target gene.

***Effect of Embodiment 1***

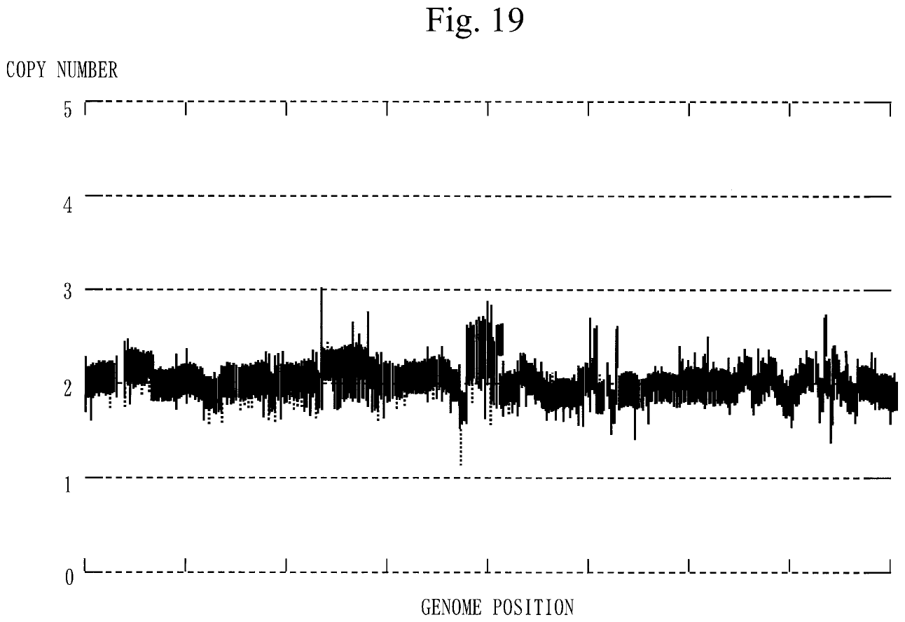

FIG. 19 illustrates the copy number in a whole genome.

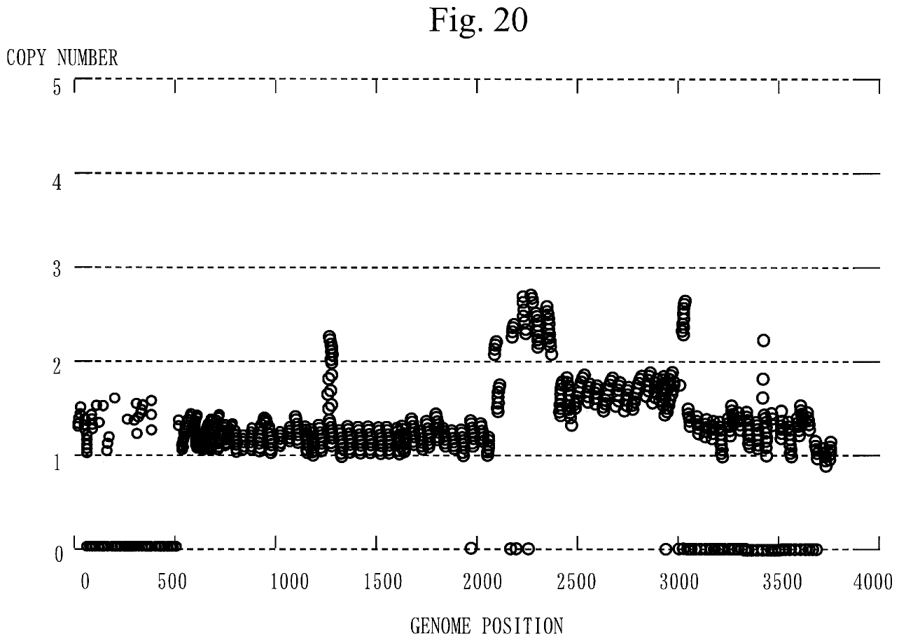

FIG. 20 illustrates the copy number of chromosome 1, the copy number of chromosome 10, and the copy number of chromosome 19.

In the whole genome (see FIG. 19), the average copy number is 2 copies. However, concerning chromosome 1, chromosome 10, and chromosome 19 (see FIG. 20) each involving a cancer-related gene, the average copy number is not 2 copies.

Ordinary CNV detection is performed supposing that the average copy number is 2 copies. Therefore, in ordinary CNV detection, the accurate copy number cannot be obtained in the target sequence.

In contrast, in Embodiment 1, by correcting the copy number, the accurate copy number can be obtained in the target sequence.

As described in Non-Patent Literature 2, a nature is known that the scatter diagram of BAF has a line-symmetric distribution with respect to the reference BAF (=0.5). This applies to the VAF as well.

In Embodiment 1, utilizing this nature, the correlation between the lower area and the upper area is found in the density distribution graph 202 derived from the scatter graph 201. Hence, the VAF in the region where this graph is obtained is obtained accurately. Thus, an accurate feature distance is obtained. As a result, the accurate copy number can be calculated.

In Embodiment 1, the accurate copy number, that is, the copy number of each target gene in the cancer cell is calculated.

Accordingly, a content ratio of the cancer cell in the tumor sample can be found.

### Embodiment 2

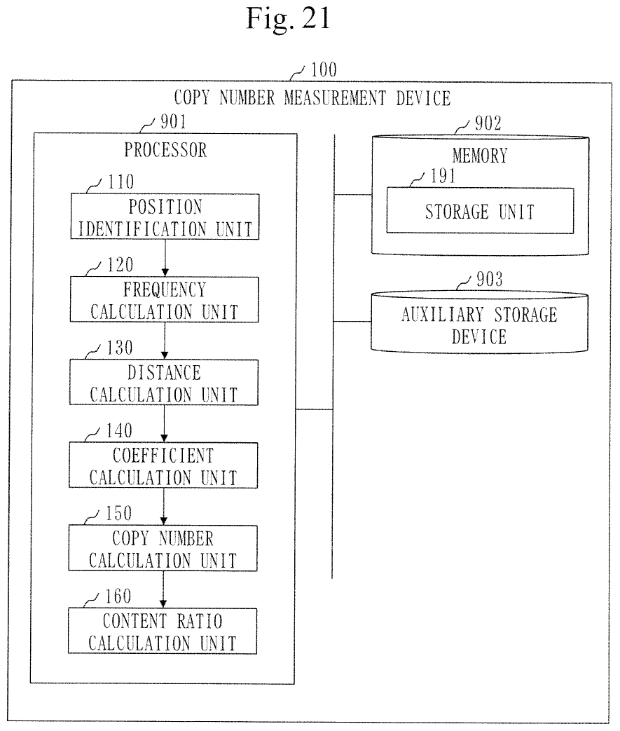

A mode to find a content ratio of a cancer cell in a tumor sample will be described referring to FIG. 21 to FIG. 23 mainly concerning differences from Embodiment 1.

***Description of Configuration***

A configuration of a copy-number measurement device 100 will be described referring to FIG. 21.

The copy-number measurement device 100 is further provided with a content ratio calculation unit 160 as a software element.

A copy-number measurement program causes the computer to further function as the content ratio calculation unit 160.

***Description of Operation***

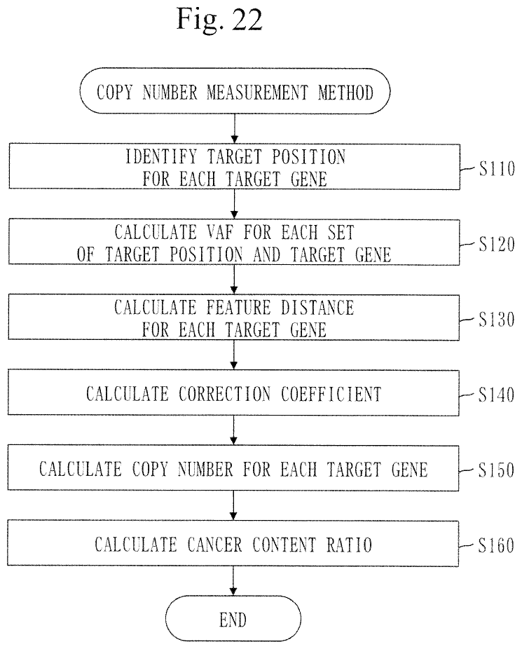

A copy-number measurement method will be described referring to FIG. 22.

Processes from step S110 to step S150 have been described in Embodiment 1 (see FIG. 2).

In step S160, the content ratio calculation unit 160 calculates a cancer content ratio based on the copy number of each target gene in a cancer cell.

The cancer content ratio is a content ratio of a cancer cell in a tumor sample.

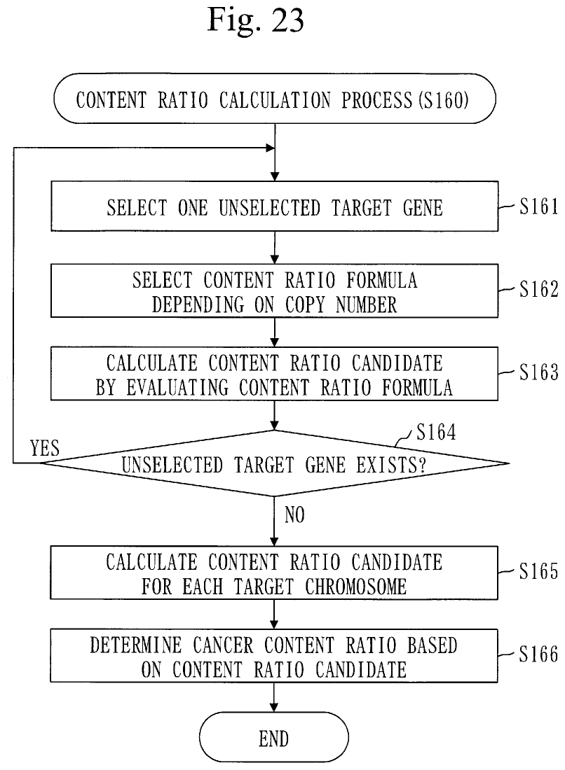

A procedure of a content ratio calculation process (S160) will be described referring to FIG. 23.

In step S161, the content ratio calculation unit 160 selects one unselected target gene.

In step S162 and step S163, a target gene signifies the target gene selected in step S161.

In step S162, the content ratio calculation unit 160 selects a content ratio formula depending on the copy number of the target gene.

The copy number of the target gene is the copy number of the target gene calculated in step S150, that is, the copy number of the target gene in the cancer cell.

A content ratio formula is a formula to find the cancer content ratio. There are two types of content ratio formulas which are a formula for LOSS and a formula for AMP. Note that LOSS signifies loss of the gene and that AMP signifies amplification of the gene.

Specifically, the content ratio calculation unit 160 selects a content ratio formula as follows.

When the copy number of the target gene is less than 2, the content ratio calculation unit 160 selects a formula for LOSS.

When the copy number of the target gene is larger than 2, the content ratio calculation unit 160 selects a formula for AMP.

In step S163, the content ratio calculation unit 160 calculates the cancer content ratio by evaluating the selected content ratio formula. The calculated cancer content ratio serves as a content ratio candidate.

Specifically, the content ratio calculation unit 160 evaluates the content ratio formula using the copy number of the target gene.

In the content ratio formulas listed below, CR expresses a cancer content ratio and CN expresses the copy number.

A formula for LOSS is:

CR=2−CN

The formula for LOSS is based on the following formula which indicates the relation between CN and CR.

CN=2(1−CR)+1×CR=2−CR

A formula for AMP is as follows. Note that n is a value estimated as the copy number in the cancer cell. When n cannot be estimated, the cancer content ratio cannot be calculated using the formula for AMP.

CR=(CN−2)/(n−2)

The formula for AMP is based on the following formula which indicates a relation among CN, CR, and n.

CN=2(1−CR)+n×CR=2+(n−2)×CR

In step S164, the content ratio calculation unit 160 determines whether an unselected target gene exists.

If an unselected target gene exists, the process proceeds to step S161.

If an unselected target gene does not exist, the process proceeds to step S165.

In step S165, the content ratio calculation unit 160 calculates a content ratio candidate for each target chromosome.

A method of calculating the content ratio candidate of the target chromosome is similar to a method of calculating a content ratio candidate of the target chromosome.

In step S166, the content ratio calculation unit 160 determines the cancer content ratio based on the content ratio candidate of each target gene and the content ratio candidate of each target chromosome.

For example, the content ratio calculation unit 160 calculates an average of the content ratio candidate of each target gene and the content ratio candidate of each target chromosome. The calculated average is the cancer content ratio.

***Effect of Embodiment 2***

With Embodiment 2, the content ratio of the cancer cell in the tumor sample can be found.

As a result, treatment suitable for the individual patient can be selected in accordance with the content ratio of the cancer cell in the tumor sample.

***Supplement to Embodiments***

The copy-number measurement device 100 may be provided with dedicated hardware devices in place of a versatile hardware device such as the processor 901. Such hardware devices are collectively called processing circuitry.

The processing circuitry implements the position identification unit 110, the frequency calculation unit 120, the distance calculation unit 130, the coefficient calculation unit 140, the copy-number calculation unit 150, and the content ratio calculation unit 160.

In the processing circuitry, one or more functions may be implemented by hardware while the remaining functions may be implemented by software or firmware. There may be one set of processing circuitry or a plurality of sets of processing circuitry.

Each embodiment is an exemplification of a preferred mode and is not intended to restrict the technical scope of the present invention. Each embodiment may be practiced partially or in combination with another embodiment. The procedure described using the flowcharts and so on may be modified appropriately.

### REFERENCE SIGNS LIST

100: copy-number measurement device; 110: position identification unit; 120: frequency calculation unit; 130: distance calculation unit; 140: coefficient calculation unit; 150: copy-number calculation unit; 160: content ratio calculation unit; 191: storage unit; 201: scatter graph; 202: density distribution graph; 203: correlation graph; 210: relation model; 901: processor; 902: memory; 903: auxiliary storage device

1. A copy-number measurement device comprising:
processing circuitry
to map a plurality of tumor sample reads which are a plurality of reads obtained from a tumor sample involving a cancer cell, to a human genome sequence, and identify, for each target gene, a target position which is a genome position of a base, the genome position having changed with respect to the human genome sequence,
to calculate a variant allele frequency for each target position of each target gene,
to calculate, for each target gene, a feature distance equivalent to a difference between a variant allele frequency corresponding to a peak density and a reference variant allele frequency in a density distribution indicating a density of a number of mapping reads with respect to the variant allele frequency, the number of mapping reads being a number of tumor sample reads mapped to respective target positions in the target gene,
to calculate a correction coefficient being used for correcting a copy number of each target gene in the tumor sample, using the feature distance of each target gene, and
to calculate a copy number of each target gene in the cancer cell using the copy number of each target gene in the tumor sample and the correction coefficient.

2. The copy-number measurement device according to claim 1,
wherein the processing circuitry generates a scatter graph indicating a relation between a variant allele frequency of each target position and the mapping read number of each target position; converts the scatter graph to a density distribution graph; generates a correlation graph indicating a correlation between a lower area and a upper area, the lower area being, of the density distribution graph, a region expressing a variant allele frequency that is equal to or lower than the reference variant allele frequency, the upper area being, of the density distribution graph, a region expressing a variant allele frequency that is equal to or higher than the reference variant allele frequency; and calculates, as the feature distance, an absolute value of a difference between a variant allele frequency corresponding to a peak correlation value and the reference variant allele frequency, in the correlation graph.

3. The copy-number measurement device according to claim 2,
wherein the correlation graph indicates a correlation in density between a variant allele frequency in the lower area and a variant allele frequency in the upper area that are equal to each other regarding absolute values of differences thereof from the reference variant allele frequency.

4. The copy-number measurement device according to claim 1,
wherein the processing circuitry calculates a value corresponding to a deviation amount between a relation graph and a measurement point, as the correction coefficient, the relation graph indicating a relation between the feature distance and a logarithmic value of a proportion of a copy number of a gene in a cancer cell to a copy number of a gene in a normal cell, the measurement point indicating a feature distance of a target gene, and a logarithmic value of a proportion of a copy number of the target gene in the tumor sample to a copy number of the target gene in a normal sample.

5. The copy-number measurement device according to claim 1,
wherein the processing circuitry calculates a content ratio of the cancer cell in the tumor sample based on a copy number of each target gene in the cancer cell.

6. The copy-number measurement device according to claim 5,
wherein the processing circuitry calculates a content ratio candidate using a copy number in the cancer cell for each target gene, and determines the content ratio of the cancer cell in the tumor sample based on the content ratio candidate of each target gene.

7. The copy-number measurement device according to claim 1,
wherein the tumor sample is a sample of a brain tumor, and
wherein the target gene is at least one of ATRX, IDH1, IDH2, TP53, TERT, BRAF, PDGFRA, MET, EGFR, BRSK1, EHD2, AKT2, TP73, NMNAT1, TGFBR3, and PTEN.

8. A non-transitory computer-readable medium storing a copy-number measurement program to cause a computer to function as:
a position identification unit to map a plurality of tumor sample reads which are a plurality of reads obtained from a tumor sample involving a cancer cell, to a human genome sequence, and identify, for each target gene, a target position, which is a genome position of a base, the genome position having changed with respect to the human genome sequence;
a frequency calculation unit to calculate a variant allele frequency for each target position of each target gene;
a distance calculation unit to calculate, for each target gene, a feature distance equivalent to a difference between a variant allele frequency corresponding to a peak density and a reference variant allele frequency in a density distribution indicating a density of a number of mapping reads with respect to the variant allele frequency, the number of mapping reads being a number of tumor sample reads mapped to respective target positions in the target gene;
a coefficient calculation unit to calculate a correction coefficient being used for correcting a copy number of each target gene in the tumor sample, using the feature distance of each target gene; and
a copy-number calculation unit to calculate a copy number of each target gene in the cancer cell using the copy number of each target gene in the tumor sample and the correction coefficient.

9. The non-transitory computer-readable medium storing the copy-number measurement program, according to claim 8,
wherein the distance calculation unit generates a scatter graph indicating a relation between a variant allele frequency of each target position and the number of mapping reads of each target position; converts the scatter graph to a density distribution graph; generates a correlation graph indicating a correlation between a lower area and a upper area, the lower area being, of the density distribution graph, a region expressing a variant allele frequency that is equal to or lower than the reference variant allele frequency, the upper area being, of the density distribution graph, a region expressing a variant allele frequency that is equal to or higher than the reference variant allele frequency; and calculates, as the feature distance, an absolute value of a difference between a variant allele frequency corresponding to a peak correlation value and the reference variant allele frequency in the correlation graph.

10. The non-transitory computer-readable medium storing the copy-number measurement program, according to claim 9,
wherein the correlation graph indicates a correlation in density between a variant allele frequency in the lower area and a variant allele frequency in the upper area that are equal to each other regarding absolute values of differences thereof from the reference variant allele frequency.

11. The non-transitory computer-readable medium storing the copy-number measurement program, according to claim 8,
wherein the coefficient calculation unit calculates a value corresponding to a deviation amount between a relation graph and a measurement point, as the correction coefficient, the relation graph indicating a relation between the feature distance and a logarithmic value of a proportion of a copy number of a gene in a cancer cell to a copy number of a gene in a normal cell, the measurement point indicating a feature distance of a target gene, and a logarithmic value of a proportion of a copy number of the target gene in the tumor sample to a copy number of the target gene in a normal sample.

12. The non-transitory computer-readable medium storing the copy-number measurement program, according to claim 8, comprising
a content ratio calculation unit to calculate a content ratio of the cancer cell in the tumor sample based on a copy number of each target gene in the cancer cell.

13. The non-transitory computer-readable medium storing the copy-number measurement program, according to claim 12,
wherein the content ratio calculation unit calculates a content ratio candidate using a copy number in the cancer cell for each target gene, and determines the content ratio of the cancer cell in the tumor sample based on the content ratio candidate of each target gene.

14. The non-transitory computer-readable medium storing the copy-number measurement program, according to claim 8,
wherein the tumor sample is a sample of a brain tumor, and
wherein the target gene is at least one of ATRX, IDH1, IDH2, TP53, TERT, BRAF, PDGFRA, MET, EGFR, BRSK1, EHD2, AKT2, TP73, NMNAT1, TGFBR3, and PTEN.

15. A copy-number measurement method comprising:
by a position identification unit, mapping a plurality of tumor sample reads which are a plurality of reads obtained from a tumor sample involving a cancer cell to a human genome sequence, and identifying, for each target gene, a target position which is a genome position of a base, the genome position having changed with respect to the human genome sequence;
by a frequency calculation unit, calculating a variant allele frequency for each target position of each target gene;
by a distance calculation unit, calculating, for each target gene, a feature distance equivalent to a difference between a variant allele frequency corresponding to a peak density and a reference variant allele frequency in a density distribution indicating a density of a number of mapping reads with respect to the variant allele frequency, the number of mapping reads being a number of tumor sample reads mapped to respective target positions in the target gene;
by a coefficient calculation unit, calculating a correction coefficient being used for correcting a copy number of each target gene in the tumor sample, using the feature distance of each target gene; and
by a copy-number calculation unit, calculating a copy number of each target gene in the cancer cell using the copy number of each target gene in the tumor sample and the correction coefficient.

16. A gene panel containing a gene set including of all of ATRX, IDH1, IDH2, TP53, TERT, BRAF, PDGFRA, MET, EGFR, BRSK1, EHD2, AKT2, TP73, NMNAT1, TGFBR3, and PTEN.

17. A gene panel containing a gene set consisting of ATRX, IDH1, IDH2, TP53, TERT, BRAF, PDGFRA, MET, EGFR, BRSK1, EHD2, AKT2, TP73, NMNAT1, TGFBR3, and PTEN.

18. A gene panel containing a gene set including at least one of ATRX, IDH1, IDH2, TP53, TERT, BRAF, PDGFRA, MET, EGFR, BRSK1, EHD2, AKT2, TP73, NMNAT1, TGFBR3, and PTEN.
